(19) United States
(12) Patent Application Publication
(10) Pub. No.: US 2014/0107446 A1
(43) Pub. Date: Apr. 17, 2014

(54) FLEXIBLE MICROELECTRODE ARRAY WITH INTEGRATED STIFFENING SHANK, AND METHOD OF FABRICATION

(71) Applicants: Vanessa Tolosa, Oakland, CA (US); Satinderpall S. Pannu, Pleasanton, CA (US); Angela C. Tooker, Dublin, CA (US); Sarah H. Felix, Oakland, CA (US); Kedar G. Shah, San Francisco, CA (US); Heeral Sheth, Oakland, CA (US)

(72) Inventors: Vanessa Tolosa, Oakland, CA (US); Satinderpall S. Pannu, Pleasanton, CA (US); Angela C. Tooker, Dublin, CA (US); Sarah H. Felix, Oakland, CA (US); Kedar G. Shah, San Francisco, CA (US); Heeral Sheth, Oakland, CA (US)

(21) Appl. No.: 14/052,639

(22) Filed: Oct. 11, 2013

Related U.S. Application Data
(60) Provisional application No. 61/802,382, filed on Mar. 16, 2013.
(60) Provisional application No. 61/713,416, filed on Oct. 12, 2012.

(51) Int. Cl. A61N 1/04; A61B 5/1468; A61B 5/04

(52) U.S. Cl. CPC A61N 1/0476; A61B 5/04; A61B 5/1468
USPC 600/345; 600/393; 29/846; 427/2.12

(57) ABSTRACT

A stiffener-reinforced microelectrode array device and fabrication method having a plurality of polymer layers surroundably encapsulating one or more electrodes connected to one or more metal traces so that the one or more electrodes are exposed. A stiffening shank is also integrally embedded in the polymer layers adjacent an insertion end of the device near the electrodes to provide mechanical support during insertion.

### CLAIM OF PRIORITY IN PROVISIONAL APPLICATION

This patent document claims the benefit and priority of U.S. Provisional Application No. 61/713,416, filed on Oct. 12, 2012, and U.S. Provisional Application No. 61/802,382, filed on Mar. 15, 2013, both of which are hereby incorporated by reference.

### FEDERALLY SPONSORED RESEARCH OR DEVELOPMENT

The United States Government has rights in this invention pursuant to Contract No. DE-AC52-07NA27344 between the United States Department of Energy and Lawrence Livermore National Security, LLC for the operation of Lawrence Livermore National Laboratory.

### FIELD OF THE INVENTION

This patent document relates to microelectrode arrays and methods of fabrication, and particularly to a microelectrode array, such as a neural interface, with an integrated stiffening shank, and methods of fabrication thereof.

### BACKGROUND OF THE INVENTION

Micro-electrode array neural probes and interfaces are essential tools in neuroscience. They provide a direct electrical interface with the neurons of a biological entity's nervous system to stimulate and/or record neural activity. Such neural probes enable researchers and clinicians to better explore and understand neurological diseases, neural coding, neural modulations, and neural topologies, and ultimately treat debilitating conditions of the nervous system, such as for example depression, Parkinson's disease, epilepsy, and deafness. In more recent years, their applications have increased from cochlear implants and pain modulation to use in more complex systems such as brain-machine interfacing and deep brain stimulation.

The most common neural probes are thin-film micromachined probes fabricated on silicon substrates using MEMS fabrication techniques. Neuronal stimulation and recording is conducted at discrete sites (metal pads) along the probes. The metal pads are connected, via metal traces, to output leads or to other signal processing circuitry. Silicon is the most widely used substrate for this type of probe because of its unique physical/electrical characteristics. The prevalence of silicon in the microelectronics industry ensures the neural probes can be relatively easily and efficiently fabricated in large numbers utilizing common MEMS fabrication techniques. There is, however, concern regarding the suitability of these silicon-based neural probes for long-term chronic) studies as the silicon will corrode over time when implanted in a body. Furthermore, the continuous micro-motion of the brain can induce strain between the brain tissue and implanted electrode promoting chronic injury and glial scarring at the implant site. Therefore, there are outstanding questions regarding the long-term safety and functionality of these silicon-based neural probes.

Polymer-based neural probes are an attractive alternative. First, they are flexible, thereby minimizing strain between the brain tissue and the implanted probe. Second, they are fully biocompatible and thus suitable for chronic implantation with no loss of functionality or safety. Finally, these polymer-based neural probes can be easily fabricated in large numbers using existing microfabrication techniques. Unfortunately, the inherent flexibility of the polymer-based neural probes means the probes also have a low mechanical stiffness causing the devices to buckle and fold during insertion. To counteract this, separate stiffening shanks are typically fabricated and then attached to individual neural probes. This procedure is very time-consuming, and in most cases, where the stiffening shanks are extremely thin (<50 μm thick), also very difficult.

As the use of MEA neural interfaces expand, the demand for multi-functionality and chronic biocompatibility also increases. As such, there is also a growing need to develop a single device capable of recording and stimulating both electrically and chemically in vivo. Most multi-electrode array (MEA) neural interfaces currently in the market or in use at academic institutions are only capable of one or two functionalities, mainly recording and stimulation of electrical signals.

Methods of drug delivery or chemical recording are often done using separate devices, in different regions of the brain if done simultaneously, and often require two different sets of equipment. This set up makes it impossible to gather electrical and chemical data from the same brain region during the same experiment. The same is true in clinical settings. Currently available neural stimulators, recorders, or drug delivery systems are all separate devices. In addition, most of these devices are constantly on or rely on timed control method, not based on the biophysical response of the body.

There is therefore a need for an improved flexible microelectrode having a stiffening shank that facilitates insertion into tissue. And there is also a need for a single device with chemical and electrical multi-functionality could provide a feedback capability that would increase the lifetime and efficacy of a medical device.

### SUMMARY OF THE INVENTION

One aspect of the present invention includes a microelectrode array, comprising: an electrically conductive layer having one or more electrodes, and one or more metal traces connected to the one or more electrodes; a plurality of polymer layers together surrounding the electrically conductive layer to at least partially encapsulate the one or more metal traces and the one or more electrodes so that the one or more electrodes are exposed; and a stiffening shank embedded in the polymer layers adjacent at least a portion of the electrically conductive layer to mechanically support said portion.

Another aspect of the present invention includes a method of fabricating a microelectrode array, comprising: forming an electrically conductive layer having one or more electrodes, and one or more metal traces connected to the one or more electrodes; forming a plurality of polymer layers to together surround the electrically conductive layer to at least partially encapsulate the one or more metal traces and the one or more electrodes so that the one or more electrodes are exposed; and forming a stiffening shank embedded in the polymer-layers adjacent at least a portion of the electrically conductive layer to mechanically support said portion.

Other aspects of the present invention include, in addition to the aspects described above for the microelectrode array, one or more of the following: a microfluidic channel formed in the polymer layers with openings therethrough leading into the microfluidic channel to communicate fluids to or from an area of interest near the one or more electrodes; a microfluidic tube connected to the polymer layers to communicate fluids to or from an area of interest near the one or more electrodes; wherein the electrodes include electrochemical sensors.

And other aspects of the present invention include, in addition to the aspects described above for the method of fabricating the microelectrode array, one or more of the following: forming a microfluidic channel in the polymer layers with openings therethrough leading into the microfluidic channel to communicate fluids to or from an area of interest near the one or more electrodes; wherein the microfluidic channel is formed by: depositing and patterning a sacrificial material for the microfluidic channel on a first one of said polymer layers, depositing a second one of said polymer layers on the sacrificial material, forming openings through the second one of said polymer layers to the sacrificial material, and releasing the sacrificial material through the openings to form the microfluidic channel; connecting a microfluidic tube to the polymer layers to communicate fluids to or from an area of interest near the one or more electrodes; and forming electrochemical sensors to the exposed one or more electrodes.

Generally, the present invention is directed to a microelectrode array having a stiffening shank integrated into its body, and an integrated, wafer-level process of fabricating the microelectrode array which incorporates the stiffening shank into its otherwise flexible body so that no post-fabrication attachment is required. With this process, polymer-based neural probes are created with a stiffened area suitable for insertion into tissue but also with a flexible cable to minimize tissue damage. Utilizing existing microfabrication techniques, large numbers of stiffened polymer-based neural probes can be created easily and efficiently. Furthermore, additional functionalities may be incorporated into the microelectrode array with integrated stiffening shank to enable additional functionalities beyond electrical sensing/recording and stimulation, such as chemical sensing, and chemical delivery as well. The general structure of the neural probes described herein have a flexible, polymer-based cable, which runs the length of the probe and contains the electrodes, interconnection traces, the stiffening shank, and optionally a microfluidic channel.

The flexible neural interface with integrated stiffening shank is suitable for implantation in both humans and animals for either acute or chronic studies of various neurological disorders and as interfaces between neural tissue and prosthetics. The neural probes described here have a flexible, polymer-based cable, which runs the length of the probe and contains the electrodes and interconnection traces and a stiffening shank at the tip (where the electrodes are located). The stiffening shank is built into the device utilizing standard microfabrication techniques, and requires no post-fabrication attachment. The flexible neural interface may be fabricated with the stiffening shank either fully encapsulated in the surrounding polymer material or partially encapsulated in the surrounding polymer material. Furthermore, these neural interfaces can be created with electrodes on the “top,” the “bottom,” or on both the “top” and “bottom.”

Further, the process is not limited to vapor-deposited (e.g. sputtering, electron-beam/thermal evaporation, atomic layer deposition, chemical vapor deposition, physical vapor deposition) materials and thicknesses.

Furthermore, the present invention provides a single device capable of multi-functionalities to improve a researcher's capability to simultaneously study multiple phenomena in the nervous system and to provide a feedback mechanism for clinical medical devices. The microfabricated multi-functional array (MFA) will be capable of electrical stimulation and recording, chemical sensing, and chemical delivery all on a minimally-sized biocompatible platform designed for in vivo implantations. One aspect of the invention includes an implantable multi-functional multi-electrode array neural interface with microfluidic channel. The polymer-based MFA described here is suitable for implantation in both humans and animals for either acute or chronic studies of various neurological disorders and as interfaces between neural tissue and prosthetics. (This assumes the materials comprising the device have been properly chosen with regards to their biocompatibility.)

In generally, the fabrication method of the present invention is independent of the array dimensions (length, width, thickness, overall shape), the electrode properties (number, spatial arrangement, thickness, shape, material), interconnection trace metal (material, thickness, shape, spatial arrangement), and the microfluidic channel dimensions (length, diameter, connections). Further, the process is not limited to vapor-deposited (e.g. sputtering, electron-beam/thermal evaporation, atomic layer deposition, chemical vapor deposition, physical vapor deposition) materials and thicknesses.

The fabrication process is also independent of the specific material used for the stiffening shank, or the thickness/dimensions of the stiffening shank. And the stiffening shank is not limited to silicon. Other materials, with varying mechanical properties, can also be used, such as other semiconductors, dielectrics (e.g. glass/quartz/silicon-dioxide, sapphire), ceramics (e.g. alumina), metals (e.g. titanium, tungsten), and others (e.g. silicon-carbide, diamond). Preferably, any material that can be etched can be used. Ultimately, the mechanical properties and the thickness of the material used dictate the stiffness of the neural interface. It is appreciated that the stiffener may be made of various types of rigid materials, including for example silicon, glass, ceramic, metal, etc. For the fully-encapsulated embodiment of the present invention, the final device will be biocompatible and suitable for chronic and acute implantation studies, regardless of whether the stiffening shank material is biocompatible (provided the chosen polymer is biocompatible). And for the partially-encapsulated embodiment of the present invention, unless the stiffening shank material is biocompatible, the neural interface created may not be suitable for chronic and/or acute implantation studies. Furthermore, various thin film MEMS fabrication methods (e.g. photolithography) may be employed to fabricate the structure of the stiffener. The stiffener fabrication process is also independent of the thickness of the stiffening shank.

The fabrication process is independent of the specific type of polymer used to create the neural interface. Polymides and parylenes (poly(p-xylylene) are the two most commonly used polymers due to their biocompatibility. Other polymers can be used (provided these materials can be deposited and etched), although these other polymers may not be biocompatible and, thus, the neural interfaces created with these materials may not be suitable for chronic and/or acute implantation studies.

### BRIEF DESCRIPTION OF THE DRAWINGS

The accompanying drawings, which are incorporated into and form a part of the disclosure, are as follows:

FIGS. 1-8 show a series of schematic cross-sectional views at various stages of fabrication of a first example multielectrode array with integrated stiffening shank of the present invention.

FIGS. 9-13 show a series of schematic cross-sectional views at various stages of fabrication following FIG. 8, of a second example multielectrode array with integrated stiffening shank of the present invention, additionally having an integrated fluidic channel, with FIG. 13 showing the multielectrode array in final form.

FIG. 14 shows a schematic top view of an insertion end of the multifunctional array produced according to FIGS. 1-13.

FIG. 15 shows the first example multielectrode array with integrated stiffening shank in final form, after removing the substrate following FIG. 8.

FIG. 16 show a schematic cross-sectional views following FIG. 15, of a third example multielectrode array with integrated stiffening shank of the present invention, additionally having an attached fluidic tubing.

### DETAILED DESCRIPTION

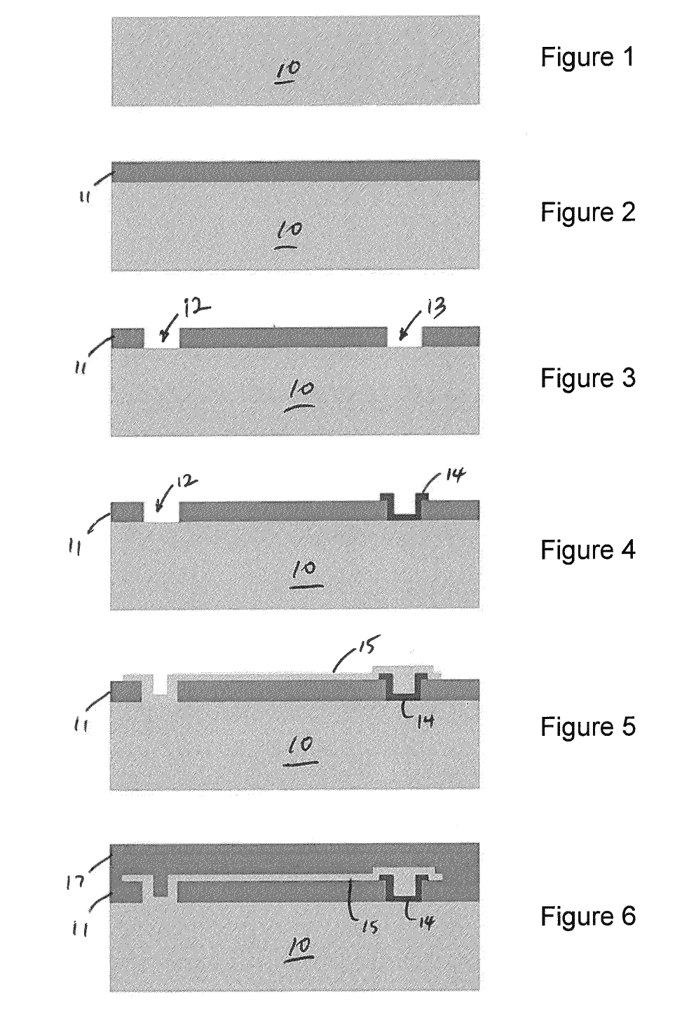

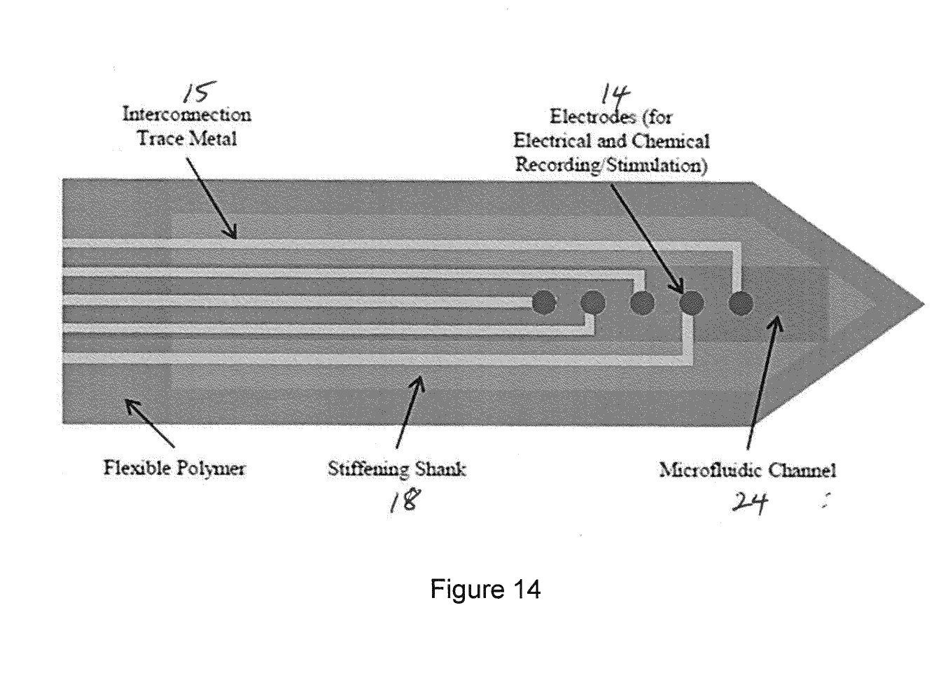

Turning now to the drawings, FIGS. 1-8 schematically show an example method of fabricating a multielectrode array with an integrated stiffening shank of the present invention, shown in final form in FIG. 15. This process creates a flexible neural interface with an encapsulated integrated stiffening shank, which may be either fully or partially encapsulated in the surrounding polymer material. For the fully encapsulated case, the material used for the stiffening shank does not need to be biocompatible, as once the fabrication process is complete, the stiffening shank is not exposed. Provided the chosen polymer for the flexible neural interface is biocompatible, the finished device will also be biocompatible and suitable for long-term implantation. In contrast, for the partially encapsulated case, the stiffening shank material is preferably selected from a biocompatible material.

As shown in particular in FIG. 1, a substrate 10 is provided, upon which a bottom polymer layer 11 is deposited in FIG. 2. Next, openings 12 and 13 are shown etched in the bottom polymer for the bottom electrode in 13, as well as for an external connector in 12. It is appreciated that opening 12 is representative of one or more openings at the connector end of the device to connect with one or more external connectors, and that opening 13 is representative of one or more openings at the insertion end of the device in which one or more electrodes are formed. In the opening 13, material for a bottom electrode 14 is deposited and patterned. Next, a bottom interconnection trace metal is deposited and patterned so as to be in contact with the electrode 14 and partially filling the opening 12. The trace metal and the electrode material together form the electrically conductive layer. Next, in FIG. 6, an interlayer polymer 17 is deposited to surround and at least partially encapsulate, together with the bottom polymer layer 11, the trade metal and the electrode 14. Next, a stiffening shank 18 is deposited or otherwise placed on the polymer layer 17. Due to the polymer layer 17, the stiffening shank is spaced adjacent the electrically conductive layer. It is notable that the stiffening shank may be deposited and patterned to extend at least a portion of the electrically conductive layer. For example, the stiffening shank may formed only at the insertion end of the device near the electrodes to provided mechanical support to the insertion end during insertion, but not along a flexible cable section of the device near a connector end where flexibility may be desirable. Furthermore it is notable that the stiffening shank may be additionally patterned so that the deposition of a next polymer layer fully encapsulates the shank. Or in the alternative the shank may be left to extend to the sides of the device so as to be exposed therealong, and thus only partially encapsulated. In either case, another interlayer polymer 19 is deposited to encapsulate the shank 18, as shown in FIG. 8. As shown in FIG. 15, removal of the substrate yields the final form of the first example microelectrode array. It is notable that while silicon may be used as the substrate material, any material can be used provided that it is compatible with the techniques and chemicals used during the microfabrication. And in some cases, a metal release layer (e.g. chrome) may be deposited on the substrate prior to the first step of the fabrication process to ensure an easy release of the final device. Furthermore, if multiple layers or interconnection trace metals are required, then after the patterning of the interconnection trace metal, the following additional steps may be employed: deposit another interlayer polymer, etch interlayer openings in the polymer, and deposit and pattern the second interconnection trace metal. And these steps can be repeated as many times as necessary to create the required number of interconnection trace metal layers.

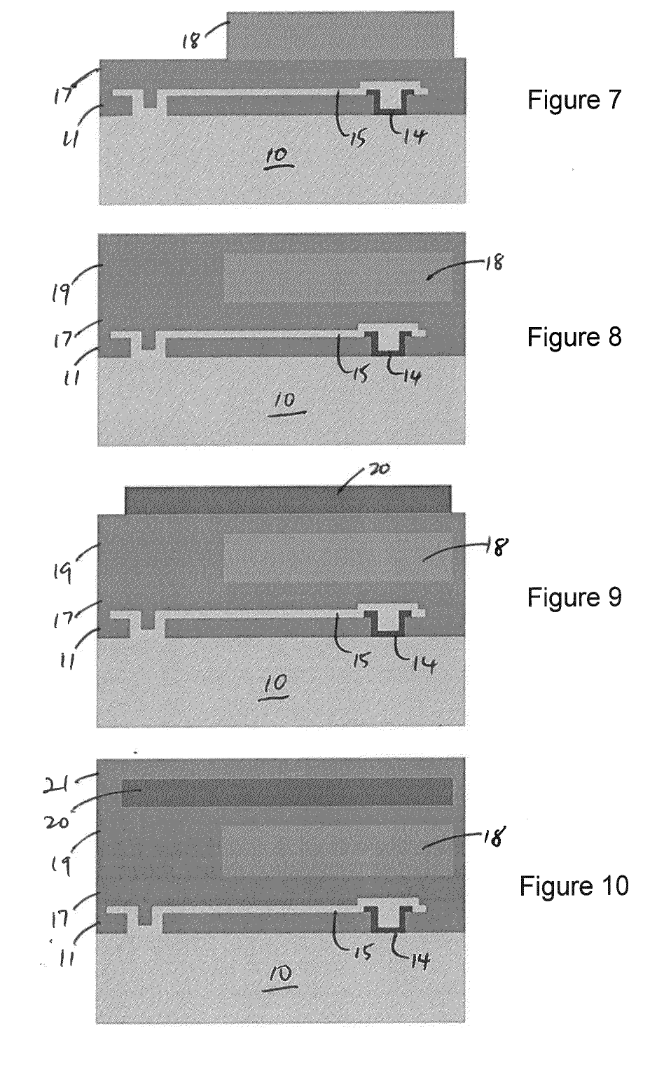

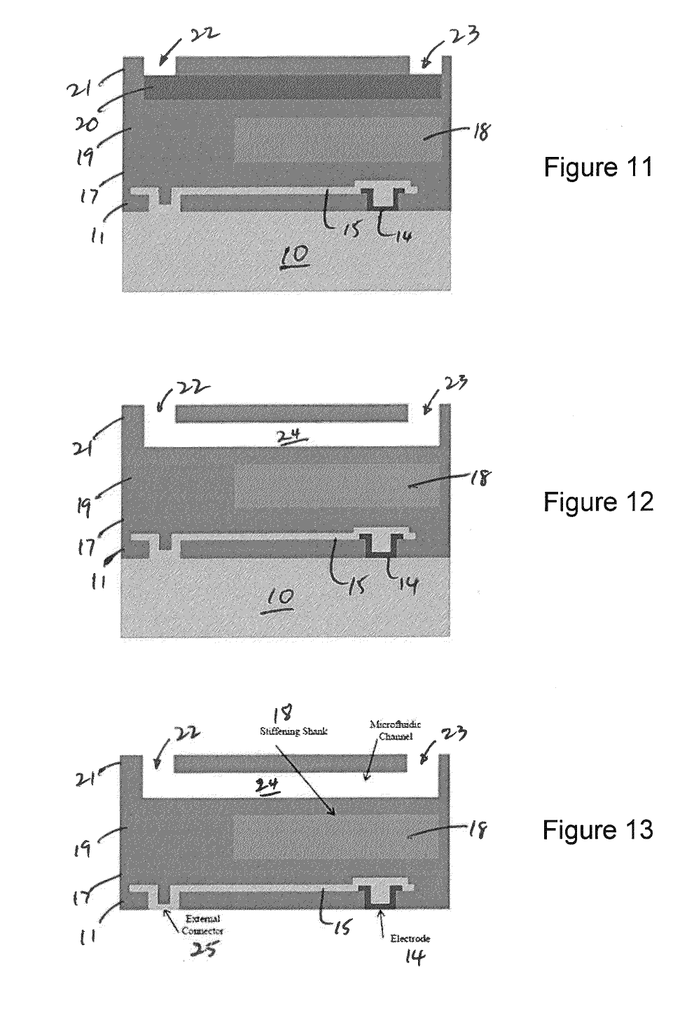

FIGS. 9-13 show a series of additional steps following FIG. 8, for fabricating an integrated microfluidic channel. In particular, a sacrificial material 20, such as photoresist material is deposited and patterned for the microfluidic channel. Next a top polymer 21 is deposited and etched to form openings 22 (e.g. inlet) and 23 (e.g. outlet) at opposite ends of the sacrificial material to form the microfluidic openings. At this point, the device outlines may be etched (including stiffening shank) to form the final shape of the device. As shown in FIG. 12, the sacrificial material 20 it then removed, (e.g. by dissolution in acetone) to form the microfluidic channel 24 in fluidic communication with openings 22 and 23. The device may be release from the substrate 10 as shown in FIG. 13. FIG. 14 shows a top schematic view of the device thus formed.

Optionally, chemical sensors may be deposited on the electrode 14 or other electrodes formed (not shown). For chemical sensing capability, electrochemical methods may be employed. Electrochemical sensing of analytes will be accomplished by applying appropriate current or voltage waveforms (including constant current and constant potential) to the sensing electrode. Sensitivity and selectivity will be optimized by varying applied waveforms and by chemically and physically modifying individual electrode sites. Sensitivity can be increased by increasing the effective surface area of the electrode sites and by reducing noise. This can be done by various physical and chemical methods including but not limited to roughening by plasma attack, using microfabrication techniques to deposit a highly porous electrode, deposition of conductive nanoparticles to increase surface area, and electroplating such that a high surface area electrode is formed. Selectivity can be improved by optimizing the applied waveforms and by size or electrostatic exclusion using semi-permeable thin film polymers. Depending on the properties of the analytes of interest and known interferents, the appropriate polymers will be deposited via dip-coating, electrochemical methods, or MEMS methods. The polymers could include but are not limited to Nafion, polypyrrole, and phenylenediamine.

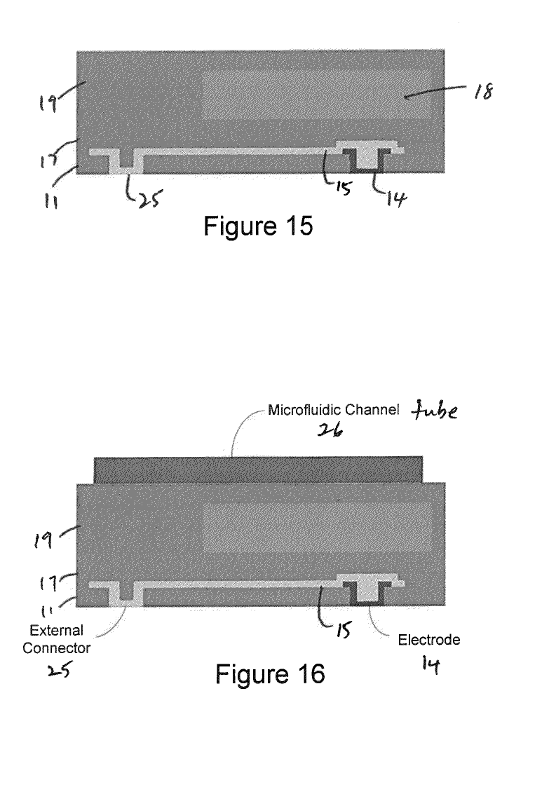

FIG. 16 shows a third example embodiment of the microelectrode array formed by attaching a microfluidic tube 26 to the shank-reinforced device of FIG. 15. Various types of adhesives, e.g. epoxy, may be utilized.

While particular operational sequences, materials, temperatures, parameters, and particular embodiments have been described and or illustrated, such are not intended to be limiting. Modifications and changes may become apparent to those skilled in the art, and it is intended that the invention be limited only by the scope of the appended claims.

We claim:

1. A microelectrode array, comprising:
an electrically conductive layer having one or more electrodes, and one or more metal traces connected to the one or more electrodes;
a plurality of polymer layers together surrounding the electrically conductive layer to at least partially encapsulate the one or more metal traces and the one or more electrodes so that the one or more electrodes are exposed; and
a stiffening shank embedded in the polymer layers adjacent at least a portion of the electrically conductive layer to mechanically support said portion.

2. The microelectrode array of claim 1, further comprising:
a microfluidic channel formed in the polymer layers with openings therethrough leading into the microfluidic channel to communicate fluids to or from an area of interest near the one or more electrodes.

3. The microelectrode array of claim 1, further comprising:
a microfluidic tube connected to the polymer layers to communicate fluids to or from an area of interest near the one or more electrodes.

4. The microelectrode array of claim 1,
wherein the electrodes include electrochemical sensors.

5. A method of fabricating a microelectrode array, comprising:
forming an electrically conductive layer having one or more electrodes, and one or more metal traces connected to the one or more electrodes;
forming a plurality of polymer layers to together surround the electrically conductive layer to at least partially encapsulate the one or more metal traces and the one or more electrodes so that the one or more electrodes are exposed; and
forming a stiffening shank embedded in the polymer layers adjacent at least a portion of the electrically conductive layer to mechanically support said portion.

6. The method of claim 5, further comprising:
forming a microfluidic channel in the polymer layers with openings therethrough leading into the microfluidic channel to communicate fluids to or from an area of interest near the one or more electrodes.

7. The method of claim 6:
wherein the microfluidic channel is formed by: depositing and patterning a sacrificial material for the microfluidic channel on a first one of said polymer layers; depositing a second one of said polymer layers on the sacrificial material; forming openings through the second one of said polymer layers to the sacrificial material; and releasing the sacrificial material through the openings to form the microfluidic channel.

8. The method of claim 5, further comprising:
connecting a microfluidic tube to the polymer layers to communicate fluids to or from an area of interest near the one or more electrodes.

9. The method of claim 5, further comprising:
further comprising forming electrochemical sensors to the exposed one or more electrodes.
